(19) United States
(12) Patent Application Publication
(10) Pub. No.: US 2006/0116583 A1
(43) Pub. Date: Jun. 1, 2006

(54) Ultrasonic diagnostic apparatus and control method thereof

(76) Inventors: Yoichi Ogasawara, Nasushiobara-shi (JP); Tetsuya Kawagishi, Nasushiobara-shi (JP); Akihiro Sano, Otawara-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/285,016

(22) Filed: Nov. 23, 2005

(30) Foreign Application Priority Data
Nov. 26, 2004 (JP) 2004-342400

(51) Int. Cl. A61B 8/14

(52) U.S. Cl. 600/458

(57) ABSTRACT

In the event that images are acquired for multiple cross-sections (multiple phases) by a predetermined imaging mode in an examination under stress to a subject, images of the same phase acquired by the same imaging mode in an examination before stress to the subject is read and simultaneously displayed as reference images together with current images. The operator can bring the cross-sectional position in the examination under stress in correspondence to the position of the examination before stress by comparing the displayed reference images with the current image.

### CROSS-REFERENCE TO RELATED APPLICATIONS

This application is based upon and claims the benefit of priority from prior Japanese Patent Application No. 2004-342400, filed Nov. 26, 2004, the entire contents of which are incorporated herein by reference.

### BACKGROUND OF THE INVENTION

1. Field of the Invention

The present invention relates to an ultrasonic diagnostic apparatus, which has an image display function to support ultrasonic image diagnosis and its control method primarily for a complicated examination combining contrast-enhanced ultrasonography and stress examination.

2. Description of the Related Art

An ultrasonic diagnostic apparatus is a diagnostic instrument, which displays images of in-vivo information and is used as a useful apparatus which is inexpensive and free of exposure to radiation as compared to X-ray diagnostic apparatus, computerized transverse axial tomography and other diagnostic imaging apparatus and enables noninvasive real-time observation. Because of such characteristics, the applicable range of an ultrasonic diagnostic apparatus is wide and is utilized for diagnosis of circulatory organs including heart, etc. to abdominal area such as liver, kidney, etc., peripheral blood vessels, obstetrics and gynecology, cerebral blood vessels, and others.

In image diagnosis using this ultrasonic diagnostic apparatus, there is an image pickup method called contrast echo. This is a method to administer an ultrasound contrast agent and allow it to flow into the pickup subject, thereby increasing ultrasonic reflection wave signals to obtain diagnostic images with higher contrast. In addition, in recent years, an ultrasound contrast agent that can be infused into the veins has been developed, and because it provides higher non-invasiveness and ensures easier handling than conventional arterial CO₂ injection, the contrast echo technique is increasingly gaining popularity. In addition, all the manufacturers are carrying forward technological developments to improve detection sensitivity of contrast agent and new imaging method.

In general, as one of the techniques to diagnose a degree of coronary occlusion, myocardial viability, etc., there is an examination by comparison evaluation of perfusion image brightness in the same cross-section before stress (cardiac load) and under stress using ultrasound contrast agent. In order to obtain effective diagnosis information by this examination, it is important to thoroughly enhance the myocardium by the contrast agent, and to obtain the same cross-section before and after (or under) stress from the viewpoint of comparison. In particular, when quantitative evaluation is carried out, it is essential to obtain the same cross-section.

With respect to the problem concerning the perfusion image of the former, attempts have been made to solve the problem by providing a function to store optimum settings of ultrasonic wave transmission sound pressure and focusing conditions, transmission and reception filter conditions, transmission intervals, transmission method, and other transmission and reception conditions in advance in the ultrasonic diagnostic apparatus, read the settings as required, and automatically set the conditions or by adjusting the contrast agent, such as concentration of contrast agent, injecting speed, etc.

On the other hand, with respect to the cross-sectional alignment of the latter, in addition to complicated examination by the use of contrast agent, examination must be completed within the limited time due to restriction of the dose of contrast agent. Consequently, in the actual diagnosis site, a technique for a physician and a engineer to memorize the cross-sectional position before stress, and based on this, decide the cross-section after stress, and carry out screening, or a technique to print images of a certain moment before stress and align the position with an estimation made at the position while referring to the printed images are adopted.

However, decision of the cross-section under stress based on artificial image memory lacks objectivity and imposes large artificial load. In addition, in deciding the cross-section after stress based on printed images, in addition to the need of printing work, there is a need to alternately move visual line between the display monitor screen and the printed image, resulting in big load to the operator.

In the cardiac contrast echo examination using an ultrasonic diagnostic apparatus, it is general practice to use two-screen display which displays an imaging mode which continuously transmits low sound-pressure ultrasonic waves with care not to destroy the contrast agent (or with care to prevent the contrast agent in the picked up cross-section from being eliminated to observe the contrast agent inflow process) (hereinafter, referred to as the “monitor mode”) and an imaging mode which intermittently transmits high sound-pressure ultrasonic waves in such a manner as to destroy the contrast agent (or to eliminate the contrast agent from the picked up cross-section while imaging the perfusion condition of the picked up cross-section by the contrast agent) (hereinafter, referred to as the “flash mode”). However, in the stress examination using the ultrasound contrast agent, modes used at the time of cross-sectional alignment or at the time of evaluation vary in accord with the pickup phases. Consequently the conventional ultrasonic diagnostic apparatus has a problem that image acquisition/display functions used in simple stress examination by one-mode one-display are unable to be appropriated as they are.

### BRIEF SUMMARY OF THE INVENTION

The present invention has been made in view of the above conditions, and it is an object of the present invention to provide an ultrasonic diagnostic apparatus that can objectively and simply choose a substantially same cross-section before and after stress and its control method, for example, in an examination by comparing and evaluating perfusion brightness in the same cross-section before stress and under stress using ultrasound contrast agent.

The present invention implements the following techniques in order to achieve the above-mentioned object.

According to an aspect of the present invention, there is provided an ultrasonic diagnostic apparatus comprising: an image acquisition unit which acquires a plurality of ultrasonic tomographic images by a first imaging mode which executes a first ultrasonic transmission that destroys a contrast agent and a second imaging mode which executes the second ultrasonic transmission that visualizes a process of flowing in of contrast agent without practically destroying the contrast agent in examination before stress and examination under stress of a subject implemented in time series; a storage unit which stores multiple ultrasonic tomographic images in each examination and in each imaging mode; an image generator unit which generates images acquired in the same imaging mode in the examination before stress as reference images in accordance with the stored multiple ultrasonic tomographic images, when current images are acquired in the predetermined imaging mode in the examinations under stress; and a display unit which simultaneously displays the reference image and the current images.

According to another aspect of the present invention, there is provided an ultrasonic diagnostic apparatus comprising: an image acquisition unit which acquires multiple ultrasonic tomographic images in accordance with multiple imaging modes in each of the multiple examinations executed in time series; a storage unit which stores the multiple ultrasonic tomographic images in accord with examinations and imaging modes; an image generator unit which generates images acquired in the same imaging mode in examinations executed in the past as reference images in accordance with the multiple ultrasonic tomographic images stored, when current images are acquired by the predetermined imaging modes in the examination under execution; and a display unit which simultaneously displays the reference images and the current images.

According to yet another aspect of the present invention, there is provided an ultrasonic diagnostic apparatus control method, comprising causing the ultrasonic diagnostic apparatus: to acquire multiple ultrasonic tomographic images in a first imaging mode which executes a first ultrasonic transmission to destroy a contrast agent and image the perfusion image condition and a second imaging mode which executes a second ultrasonic transmission to image the process in which the contrast agent flows in without practically destroying the contrast agent; to store the multiple ultrasonic tomographic images in accord with examinations and imaging modes; to generate images acquired by the same imaging mode in the examination before stress as reference images in accordance with the multiple ultrasonic tomographic images stored when current images are acquired by the predetermined imaging mode in the examination under stress; and to simultaneously display the reference images and the current images.

According to yet another aspect of the present invention, there is provided an ultrasonic diagnostic apparatus control method, comprising causing the ultrasonic diagnostic apparatus: to acquire multiple ultrasonic tomographic images in accordance with multiple imaging modes in each of the multiple examinations executed in time series; to store the multiple ultrasonic tomographic images in accord with examinations and imaging modes; to generate images acquired in the same imaging mode in examinations executed in the past as reference images in accordance with the multiple ultrasonic tomographic images stored, when current images are acquired by the predetermined imaging modes in the examination under execution; and to simultaneously displays the reference images and the current images.

According to yet another aspect of the present invention, there is provided an ultrasonic diagnostic apparatus control method which comprises: acquiring a first image according to a first imaging mode and a second image according to a second image respectively in a first examination; storing the first image and the second image according to imaging mode; displaying the first image and a third image simultaneously when the third image is acquired according to the first imaging mode in a second examination which is performed after the first examination; displaying the third image and a fourth image simultaneously when the fourth image is acquired according to the second imaging mode in the second examination; and displaying the second image and the fourth image simultaneously when the second examination is completed.

### BRIEF DESCRIPTION OF THE SEVERAL VIEWS OF THE DRAWING

FIG. 1 is a block diagram of an ultrasonic diagnostic apparatus 1 according to the present embodiment;

FIGS. 2A to 2D are illustrations that explain image storage function, image reference function, and image comparing function, and shows one example of display form of a monitor 14 in the contrast echo technique;

FIG. 3 is a flow chart that indicates a flow of each processing executed before stress of the contrast echo technique;

FIG. 4 is a flow chart that indicates a flow of each processing executed under stress of the contrast echo technique;

FIG. 5 is a diagram that indicates a reference image (left) and a current image (right) displayed on the monitor 14 by the image reference function;

FIG. 6 is a diagram that indicates a flow chart that indicates a flow of each processing executed in accordance with image comparison function;

FIG. 7 is an illustration that indicates a still image (left) by the flash mode before stress synthetically displayed on the monitor 14 by the image comparison function and a still image (right) by the flash mode under stress;

FIG. 8 is an illustration that indicates one example of edit screen of workflow;

FIG. 9 is an illustration that indicates one example of workflow W, which defines stress examination concerning ischemic heart diseases;

FIG. 10 is a flow chart that indicates a flow of each processing executed in accordance with the image comparison function of the ultrasonic diagnostic apparatus 1;

FIG. 11 is an illustration that indicates one example of tomographic images before and after stress displayed on the monitor 14 by the image comparison function; and

FIG. 12 is an illustration that indicates another example of tomographic images before and after stress displayed on the monitor 14 by the image comparison function.

### DETAILED DESCRIPTION OF THE INVENTION

Referring now to drawings, first to third embodiments according to the present invention will be described in detail as follows. In the following description, reference symbols designate substantially same or corresponding parts throughout, and redundant explanation will be only made whenever necessary.

### First Embodiment

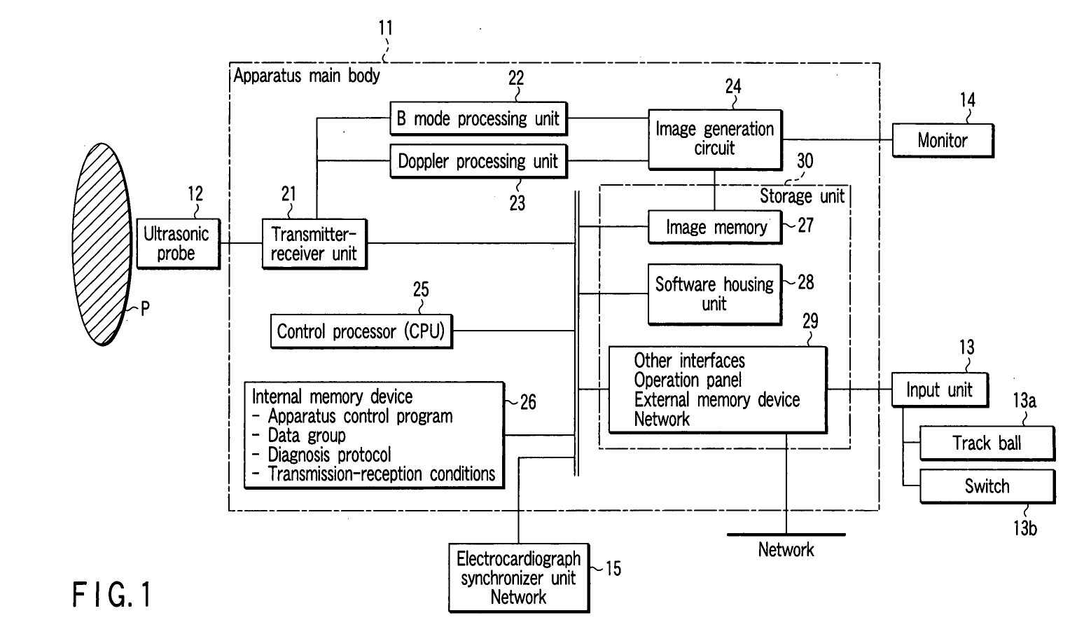

FIG. 1 is a block diagram of an ultrasonic diagnostic apparatus 1 according to the present embodiment. As shown in the drawing, the ultrasonic diagnostic apparatus 1 comprises an ultrasonic probe 12, apparatus main body 11, external input unit 13 that is connected to the apparatus main body 11 and imports various directions, orders, and information from the operator into the apparatus main body 11, and a monitor 14 which is an external display unit to display images created by the apparatus main body 11. To the input unit 13, a track ball 13a, switch 13b, etc. are installed to set a region of interest (ROI).

The ultrasonic probe 12 has a piezoelectric vibrator as an acoustic/electric reversible conversion element such as piezoelectric ceramic. Multiple piezoelectric vibrators are arranged in a line and mounted to the head end of the probe 12.

The apparatus main body 11 is connected to other units, internal memory device 26, and storage unit 30 by the bus as with control processor (CPU) 25 a control center of the whole system. The storage unit 30 comprises memory 27, software storage unit 28, and other interfaces 29, etc.

An ultrasonic transmitter-receiver unit 21 reads transmission-reception conditions stored in the internal memory device 26 by control processor 25 and generates pulses in accordance with the transmission-reception conditions. In the ultrasonic transmitter-receiver unit 21, ultrasonic waves are focused in a form of beams and give each rate pulse the delay time necessary to determine the transmission directivity, and apply voltage pulses to each channel of the probe 12 in the timing which the ultrasonic transmitter-receiver unit 21 received the rate pulses. By this, the ultrasonic beams are transmitted to the subject.

In addition, in circulatory organs, in order to acquire images by specific cardiac time phase, to the apparatus main body 11, an electrocardiograph synchronizer unit 15 is connected, and electrocardiographic signals of a patient are inputted via the electrocardiograph synchronizer unit. To this electrocardiographic signal, information on displays and image acquisition timing is inputted from the operation panel 29. In the control processor 25, based on the electrocardiographic signals and the above information, image acquisition timing control is carried out on the transmitter-receiver unit 21. In addition, the electrocardiographic signal is stored in memory 27 and combined with additional information such as other image information, character information, and others in the image generation circuit 24.

In addition, based on mode selection, ROI setting, transmission start/end inputted from the user input unit 13 or other interface 29, transmission and reception conditions and apparatus control programs stored in the internal memory device 26 are read and in accordance with these, by the control processor 25, the transmitter-receiver unit 21 is controlled.

On the other hand, ultrasonic beams applied to the subject inside for image generation is reflected by the discontinuous surface of acoustic impedance in the subject and the reflected wave is received by the probe 12. The echo signal outputted from each channel of the probe 12 is incorporated in the transmitter-receiver unit 21. The echo signal is amplified for each channel in the transmitter-receiver unit 21, is given delay time necessary to determine the reception directivity, and added. By this addition, reflection components from the direction that correspond to the reception directivity are emphasized. By this transmission directivity and reception directivity, comprehensive directivity of ultrasonic transmission and reception is determined. This directivity is generally called “scanning line.”

The echo signal outputted from the transmitter-receiver unit 21 is sent to a B mode processing unit 22 and a Doppler processing unit 23. The B mode processing unit 22 comprises a not-shown logarithmic converter, envelope detector circuit, and analog digital converter (A/D). The logarithmic converter logarithmically converts echo signals. The envelope detector circuit detects the envelope of output signals from logarithmic converter. This detection signal is digitized via the analog digital converter and outputted as the detection data. In addition, the Doppler processing unit 23 extracts blood flow components using the analysis results by frequency analysis or filters, and finds the mean velocity, variance, power, and other blood flow information for multipoints.

The image generation circuit 24 performs frame correlation processing, coordinate conversion, etc. using the detection data inputted from the B mode processing unit 22 and generates B mode images. In addition, the image generation circuit 2 creates the mean velocity image, variance image, power image, and combined image of these using the blood flow information inputted from the Doppler processing unit 23. The prepared image information, etc. are sent to the monitor 14 and displayed in a predetermined form.

In the image generation circuit 24 displays the same kind of ultrasonic tomographic images obtained in the mode same as the imaging mode currently in execution on the basis of the control information later described, etc. in the stress examination concerning, for example, ischemic heart disease (hereinafter, simply referred to as “stress examination”) simultaneously (for example, in parallel) with current images. In particular, the image generation circuit 24 reads images (moving images or still images) before stress which correspond to each mode at the time of image acquisition in the monitor mode under stress in the stress examination or flash mode under control of the control processor 25 as reference images, and creates parallel images to be displayed simultaneously with images currently picked up.

The control processor 25 stores data in memory 27 or external memory device 29 on the basis of the information inputted from these various switches and track ball 13a or using the data stored, executes programs read from the software storage unit 28.

The internal memory device 26 stores control programs of the relevant apparatus, diagnosis protocols, transmission and reception conditions and other various data groups, acquired image data, etc. In particular, the internal memory device 26 stores each image acquired for each imaging mode before stress or under stress in the stress examination later described together with control information. Now, the control information includes the path name of storage place of each image, file name or folder name, imaging mode, image pickup phase, and other ancillary information, and information that correlates images acquired by the same imaging mode.

In addition, the internal memory device 26 stores control programs for controlling the apparatus in accordance with various minor programs (activities) that realize each processing that configures a series of examination procedures and the examination procedure (workflow) configured by various activities, which are described in, for example, Jpn. Pat. Appln. KOKAI Publication No. 2001-137237.

The image memory 27 temporarily stores the data, which the image generation circuit 24 handles.

The software storage unit 28 stores various programs necessary for ultrasonic diagnosis (for example, image acquisition, image processing, image display, etc.). In addition, the software storage unit 28 stores image storage function programs that realize image preserving functions, reference function programs to realize image reference functions, comparison function programs, etc. to realize image comparison functions. By reading these programs in the predetermined timing and developing into memory not illustrated, each function can be realized under the control of the control processor 25. These reference function programs, etc. may be configured to store in the internal memory device 26. The detail of each function will be described later.

The monitor 14 displays in vivo morphologic information and blood flow information as images in accordance with video signals from the image generation circuit 24. Images, etc. displayed on this monitor are saved in, for example, internal memory device 26 by the use of corresponding image data that is stored in an image capture circuit in the image generation circuit 24 or image memory 27.

To the switch 13b of the input unit 13, the following various switches are provided to control the timing to store images and display the stored images as reference, image display for reference displayed, comparison image display, etc.

Each of k pieces of phase switches SW1-1 to SW1-k supports multiple pickup phases. The image pickup phase means the phase to image the predetermined imaging cross-section in a series of imaging sequence. For example, when three cross-sections of apical four-chamber view, apical two-chamber view, and parasternal short-axis view are taken in a series of imaging sequence, three image pickup phases exist. In the embodiment, the case in which three cross-sections of apical four-chamber view, apical two-chamber view, and parasternal short-axis view are taken in a series of pickup sequence is taken as an example. Each phase switch operates exclusively from other image pickup phases and pressing the same switch changes over between ON and OFF.

The acquisition switch SW2: A switch to acquire images and by pressing this switch, image acquisition is carried out. During the acquisition time while switch SW2 is in operation, moving images or still images or both for the time set by the preset function in advance are acquired, but the setting can be optionally changed. By pressing this acquisition switch SW2, the control information on the acquired images is created and automatically stored in the internal memory device 26 together with the acquired images.

Image reference function switch SW3: A switch to give ON/OFF directions of the image reference function later described.

Image comparison function switch SW4: A switch to give ON/OFF directions of the image comparison function later described.

Reference/comparison image selector switch SW5: A switch to select an imaging mode of reference image or comparison image when image reference function or image comparison function is implemented. That is, when the image reference function switch SW3 is turned ON, selecting the monitor mode (or flash mode) by the reference/comparison image selector switch SW5 allows moving images to be displayed on the monitor 14 as reference images picked up by the monitor mode (or flash mode) before stress. In addition, selecting the monitor mode (or flash mode) by the reference/comparison image selector switch SW5 with the image comparison function switch SW4 turned ON displays two dynamic images taken by the monitor mode (or flash mode) before stress and under stress on the monitor 14 as comparison images. The reference/comparison image selector switch SW5 may change over the image mode between the monitor mode and the flash mode every time when it is pressed.

Current image selector switch SW6: A switch to select whether the image currently being taken (current image) is acquired as a moving image or a still image. The current image selector switch SW6 is configured to change over the designation of moving image and still image every time when it is pressed. Incidentally, the image mode switch SW5 and image selector switch SW6 operate as toggle switches.

State switch SW7: A switch to designate the state which exerts stress on the subject (under stress). In the embodiment, when the state switch SW7 is turned ON, the subject is under stress and when it is turned OFF, the subject is not under stress.

The reference/comparison image selector switch SW5, and the imaging mode and moving image or still image of the current image selector switch SW6 which are first designated when the image reference function switch SW3 and image comparison function switch SW4 are pressed is assigned as designated as that set by the present function in advance.

(Medical Background)

Now, description will be made on the medical background concerning the use of the present ultrasonic diagnosis apparatus.

In general, an ultrasonic examination method in ischemic heart disease adopts a technique to observe cardiac wall motion (movement of endocardium) or changes in wall pressure (degree of increase in distance between endocardium and epicardium) but in recent years, by the use of transvenous ultrasound contrast agent, a cardiac contrast echo technique (hereinafter, simply referred to as a “contrast echo technique”) has been enabled, which can visualize ischemia itself, that is, whether or not the blood flows into the myocardium, and if it flows in, at what velocity it flows into the myocardium, by obtaining signals of the contrast agent. In particular, in the case of effort angina, under the condition in which no load is exerted to the heart, symptoms of palpitation, shortness of breath, dizziness, etc. are not exhibited, but under high load such as physical exercise, that is, under stress, various symptoms are exhibited, and to diagnose this, stress examination is carried out by the use of exercise load using a tread mill or drug load by vasodilator, etc. The present ultrasonic diagnosis apparatus 1 provides a technique to support the contrast echo technique by stress.

When this contrast echo technique is practiced, by electrocardiograph synchronization, transmission is suspended, for example, for a period from 1 heartbeat to 8 heartbeats, and micro-bubbles as a contrast agent, are stored in the myocardium; then, high sound pressure transmission is carried out at the concentric time phase such as at the endsystole or at the end diastole, micro-bubbles in the blood flow are destroyed and visualized, and comparison and evaluation are carried out based on the brightness information of perfusion image, and thereby diagnosis is performed. While bubbles are stored and high sound pressure transmission is carried out for this visualization (intermittent period), the physician and the engineer must hold the cross-section and in several current ultrasonic diagnostic apparatuses, a mode (monitor mode) to transmit sound at low pressure with care to prevent micro-bubbles from being destroyed and to visualize it to hold a cross-section is offered.

In order to confirm the perfusion image, the examination is frequently carried out while two screens are being displayed and for example, on the left half of the monitor screen, an image obtained in the flash mode (flash image) and on the right half of the monitor screen, an image obtained in the monitor mode (monitor image) is being displayed.

In general, in the case of a patient suffering from ischemic heart disease with coronary artery stenosis, when the blood flow velocity in the myocardium is increased by applying load to the heart by physical exercise, blood on the ischemia portion flows to the normal portion (subclavian steal syndrome), and inflow of blood into the myocardium of the ischemic portion decreases, and sufficient motor ability is unable to be achieved. In the contrast echo technique, making the best of this subclavian steal syndrome, images in the perfusion mode are acquired before stress and under stress, and perfusion image brightness is compared before stress to that under stress in the same cross-section. By this comparison, it becomes possible to judge the portion ischemic where the perfusion image brightness lowers from that before stress, whereas it is possible to diagnose it normal when the perfusion image brightness rises.

What is important in this contrast echo technique is to compare the same cross-section, or cross-sections in the range where the same coronary artery governs, before stress and under stress. Consequently, a function to align cross-sections before stress and under stress and to display images for final comparison.

(Image Reserving Function/Image Reference Function/Image Comparison Function)

Next description will be made on the image reserving function, image reference function, and image comparison function, which the ultrasonic diagnostic apparatus 1 possesses and which is used in the contrast echo technique.

The image reserving function is a function to save images under pickup in response to the predetermined operation (for example, operation of acquisition switch SW2) and at the same time, to generate and save control information on the saved image. In addition, the image reference function is to read the image before stress corresponding to the current imaging mode in accordance with the control information and display this image as reference image dynamically or statically in order to secure the cross-section same as the cross-sectional position before stress in the monitor mode/flash mode image pickup under stress. Furthermore, the image comparison function is to display selected images before stress and selected images under stress in parallel in order to compare images.

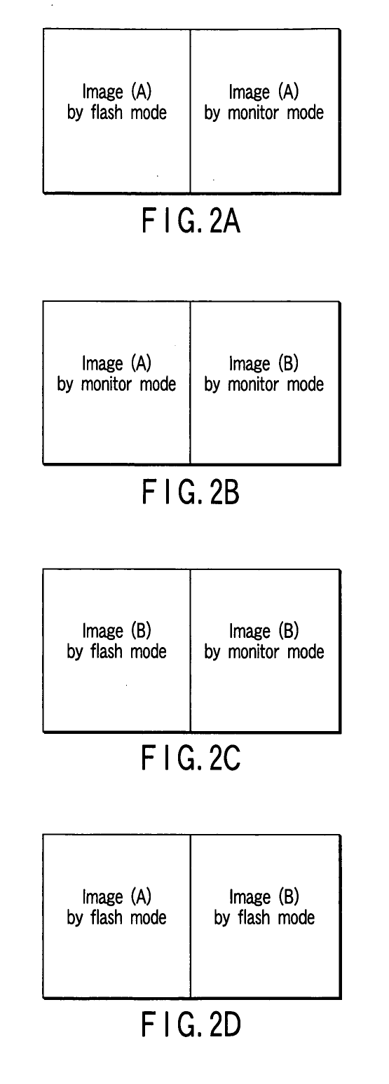

FIGS. 2A to 2D are illustrations that explain image storage function, image reference function, and image comparing function, and shows one example of display form of a monitor 14 in the contrast echo technique.

Before stress, as shown in FIG. 2A, images (A) by the flash mode and images (A) by the monitor mode, which are acquired in a predetermined timing, respectively, in synchronism with the electrocardiograph synchronizer unit, are displayed in a row (FEI DUAL display). Incidentally, reference symbol (A) for the image indicates before stress. For example, when the acquisition switch SW2 is operated under this condition, the image (A) in the monitor mode is saved and at the same time, the control information concerning the image (A) by the monitor mode is created and automatically saved.

Then, in the image pickup under stress, as shown in FIG. 2B, the image (A) in the monitor mode acquired before stress is read as a reference image to align the cross-sections in accordance with the control information and is displayed together with the image (B) in the monitor mode which is currently being taken. The operator can align the cross-sectional position of image (B) to that of image (A) by confirming the image (B) in the monitor mode referring to the image (A) in the monitor mode. Reference symbol (B) for the image indicates that the image is taken before stress.

When the cross-sectional position in image pickup under stress is determined, as shown in FIG. 2C, image (B) by the flash mode and image (B) by the monitor mode, which is acquired in a predetermined timing, respectively, in synchronism with the electrocardiograph synchronizer unit, are displayed in a row (FEI DUAL display). For example, when the acquisition switch SW2 is operated under this condition, the image (B) in the monitor mode is saved and at the same time, the control information concerning the image (A) by the monitor mode is created and automatically saved.

In addition, as shown in FIG. 2D, the image before stress and the image under stress are displayed in a row by the image comparison function in accordance with the control information so that comparison can be easily made between before-stress and under-stress.

In the example of FIG. 2D, a case in which the images (A) by the flash mode and images (B) by the monitor mode are displayed in a row is shown. The operator can observe how the cardiac perfusion in the myocardium takes place throughout the period before and after stress by comparing the image (A) by the flash mode to the image (B) obtained in the same imaging mode.

(Operation)

Next description will be made on the image-pickup operation in the contrast echo technique of the ultrasonic diagnostic apparatus 1. In the embodiment, to specifically explain, cases of taking images of three kinds of cross-sections, namely, apical four-chamber view, apical two-chamber view, and parasternal long-axis view, will be taken for example.

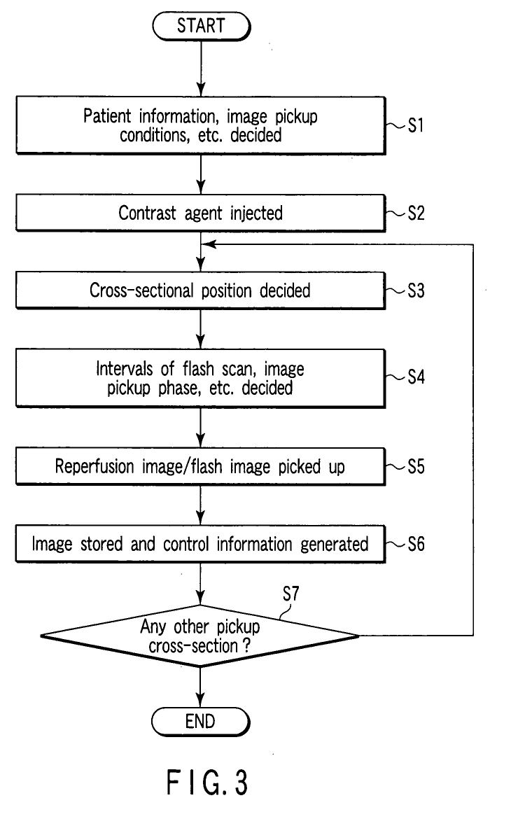

FIG. 3 is a flow chart that indicates a flow of each processing executed before stress of the contrast echo technique. As shown in the figure, first of all, patient information, various image pickup conditions, etc. are inputted (step S1).

Then, contrast agent were continuously infused into left forearm or continuously infused after bolus-injection (step S2). The display form of the monitor 14 in such a case should be to display a still image obtained by the flash mode on the left side of screen and a moving image obtained by the monitor mode on the right side of screen (FEI DUAL display). The screen display position of the two modes may be reversed. The operator determines the cross-sectional position (in the present case, the position of apical four-chamber cross section) while observing these images (step S3).

Then, in the two-mode screen display condition, the operator set the timing to carry out flash scanning, time interval and image pickup phase to carry out flash scan (step S4).

That is, the operator sets timing by the predetermined setting screen so that flash-scan can be implemented in synchronism with the cardiac time phase subject to diagnosis, for example, late in systole, in accordance with the electrocardiograph signals from the patient obtained by the electrocardiograph synchronizer unit 15. In addition, the operator scans the apical four-chamber cross-section determined in step S3 and presses the phase switch SW 1-1 to define the image pickup of the relevant cross-section as the image pickup phase 1. Now, let the scan implementation timing in which flash-scan interval is executed once per n heartbeats be referred to as “1:n”; then, the operator changes the intermittent intervals by electrocardiograph synchronizer unit by the predetermined entry screen in such a manner 1:1, 1:2, 1:3, 1:4, and so forth after pressing the phase switch SW 1-1, and when the perfusion image is successfully confirmed, the operator sets the desired flash scan implementation intervals. In this event, assume that 1:1 is selected for phase 1.

After setting the flash scan intervals, image pickup by the flash mode in 1:1 scan intervals as well as in the monitor mode is carried out (step S5). The operator presses the acquisition switch 2 when he or she confirms necessary images, and saves moving images and still images (step S6). In such a case, control information concerning the image to be saved is created and saved, too.

Each image is controlled and saved as image data, which differs for each phase by the designation of phase switch. In addition, the acquisition switch 2 may be pressed any times in each phase. When the acquisition switch 2 is pressed multiple times in the same phase, the image data is saved in chronological order in accordance with the pressing order in memory 27 or saved in external memory device 29 under an identifiable file name or in a different folder.

This completes scan concerning the apical four-chamber cross-section before stress and judgment is made on whether or not there is a next image pickup cross-section (step S7). In the embodiment, in order to pick up images of the apical two-chamber view, processing of steps S3 to S6 is carried out again as image pickup phase 2. In addition, in the same manner for the parasternal short-axis cross-section, processing of steps S3 to S6 is carried out again as image pickup phase 3.

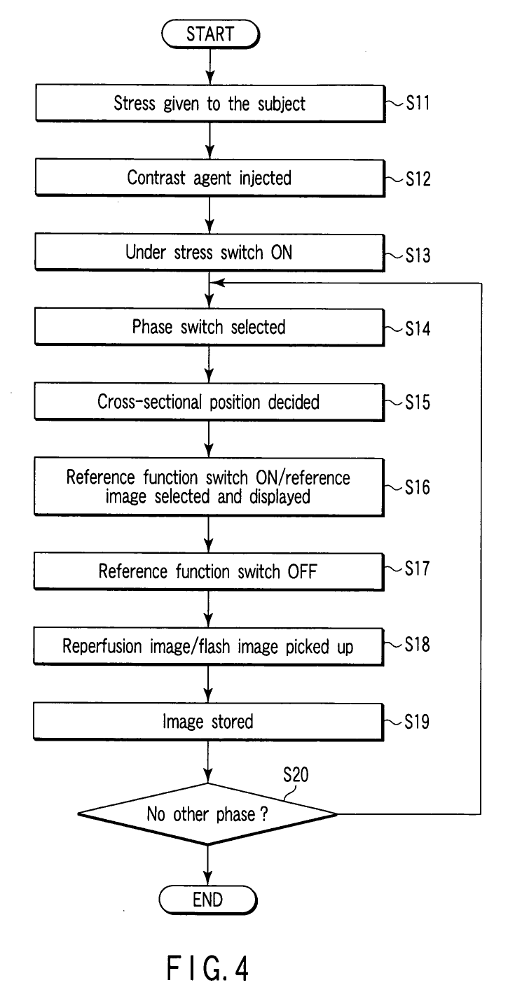

Referring now to FIG. 4, each processing implemented under stress in the contrast echo technique will be described. First of all, as shown in the same figure, stress is applied to the subject (step S11). As a method to apply stress, a technique to allow the subject to actually perform exercise or to apply load (stress) to heart by injecting a specific drug can be adopted.

The, same as the case of step S2, contrast agent is continuously infused into the left forearm or is continuously infused after bolus-injection (step S12). In such a case, display form of the monitor 14 should be FEI DUAL display.

Then, in order to designate the current image pickup as under-stress, the status switch 7 is switched on(step S13). Thereafter, images acquired with the status switch 7 pressed are handled as images under stress.

Then, the operator chooses the phase switch to designate the image pickup phase while observing the displayed images (step S14). In this case, assume that the phase switch SW 1-1 is chosen.

Then, in order to display a reference image used to align the cross-sectional position before stress to the cross-sectional position of an image to be taken now, the image reference function switch SW3 is pressed (step S15). By this, the reference function program is called from the software storage unit 28, and under the control of the control processor 25, based on the control information, the moving image of the monitor mode in the image pickup phase 1 saved before stress (that is, moving image of the apical four-chamber view) is read to be combined into the left half of the screen by the image generation circuit 24 and displayed on the monitor 14. The operator determines the image pickup cross-sectional position under stress by aligning the live image of the monitor mode displayed on the right half of the screen while confirming the moving image of the monitor mode at the time of control, which is displayed on the left half of the screen (step S16).

In the embodiment, when the image reference function switch SW3 is pressed, the reference/comparison image selector switch SW5 should be set to the monitor mode and the current image selector switch SW6 should be set to designate a dynamic image by presetting.

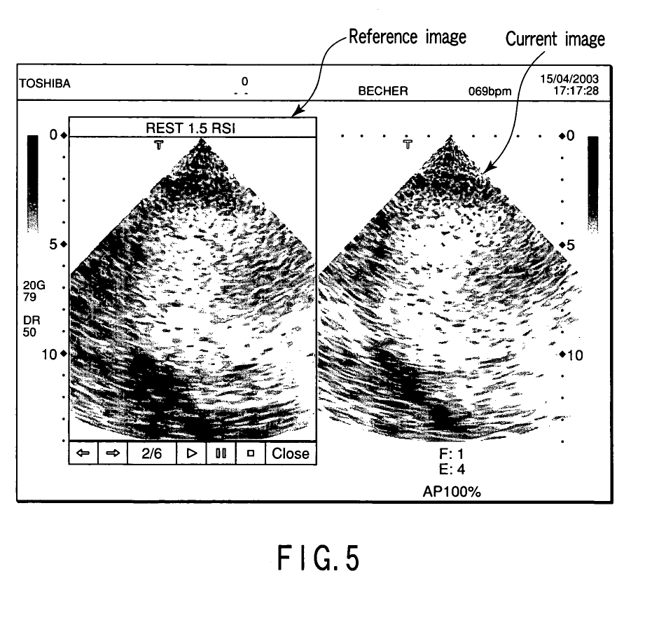

FIG. 5 is a diagram that indicates a reference image (left) and a current image (right) displayed on the monitor 14 by the image reference function. FIG. 5 corresponds to the display form shown in FIG. 2B.

As shown in FIG. 5, under the reference image, switches to choose reference images from these images when multiple images are acquired in the same phase in image pickup before stress, control switches for order display of current display image for overall acquired images, reproduction at the time of selecting moving images, temporary stop, and stop, switch to end windows in which reference images are indicated are displayed. The operator moves above the desired switch, etc. by the track ball 13a and other input unit 13, and can carry out various operations using decision switches close to the track ball.

When the cross-sectional position is determined, the operator presses the image reference function switch SW3 and ends the reference image display (step S17), and when a stable image is drawn, the operator presses the acquisition switch SW2 to take a moving image or still image of the monitor mode and flash mode in the image pickup phase 1 under stress (step S18). The obtained images are controlled in accordance with phases and modes and saved in the internal memory device 26, external memory device 29, etc. (step S19).

In image pickup under stress, too, the acquisition switch 2 and image reference function switch SW3 may be pressed freely any times as is the case of before-stress. In addition, by repeatedly pressing the acquisition switch SW2, moving image and still image for each mode to be saved in a plurality are recorded under file name or folder name that corresponds to the order in which the switch is pressed.

Next, whether or not image pickup under stress concerning other image pickup phases (for example, image pickup phase 2 concerning apical two-chamber cross-section) should be carried out, and if executed, each processing of steps S14 to S19 is repeated. On the other hand, if it is not executed, processing of contrast echo technique under stress is ended.

The foregoing technique is effective where the dosage is controlled to increase the degree of stress stepwise in loading drugs such as Dobutamine. In such a case, a configuration to implement each processing in each stage should be adopted.

(Operation in Image Comparison)

Next, description will be made on operation in compliance with the image comparison function of the ultrasonic diagnostic apparatus 1. Description will be made on cases in which each image of apical four-chamber view, apical two-chamber view, and parasternal short-axis view obtained by the image pickup method by contrast echo technique described above is compared before and after stress.

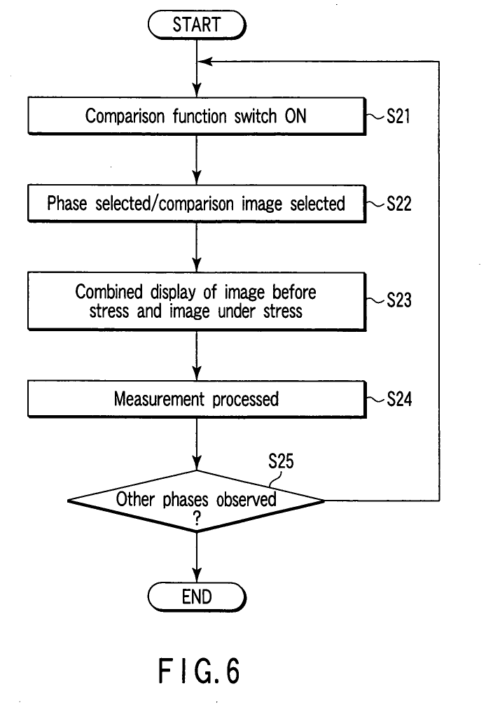

FIG. 6 is a diagram that indicates a flow chart that indicates a flow of each processing executed in accordance with image comparison function. As shown in the figure, pressing the image comparison function switch SW4 calls the program for comparison function from the software storage unit 28 in response to this operation, and under control of the control processor 25, the relevant image comparison function is started (step S21). Thereafter, the phase switch SW1-m is pressed to choose a desired image pickup phase m (m is a natural number equal to or not more than k.) subject to image comparison (step S22). In such a case, pressing the image comparison function switch SW4 in the state continued from the data acquisition under stress automatically sets the image pickup phase that corresponds to the currently active phase switch as the subject of image comparison.

When the image pickup phase subject to image comparison is chosen, images that correspond to the image pickup phase chosen on the basis of control information are read, and synthesized image is created in the image generation circuit 24 in such a manner that, for example, a still image by the flash mode before stress is displayed on left half of the screen and a still image by the flash mode under stress is located on the right half of the screen, and is displayed on the monitor 14 (step S23).

In the embodiment, it has been initially set in such a manner that when the image comparison function switch SW4 is pressed, still images by the flash mode before stress and under stress are displayed. However, the present invention is not intended to be limited to this but for example, by the settings using the reference/comparison image selector switch SW5 and current image selector switch SW6, images can be compared between different imaging modes, between dynamic image and still image, and others.

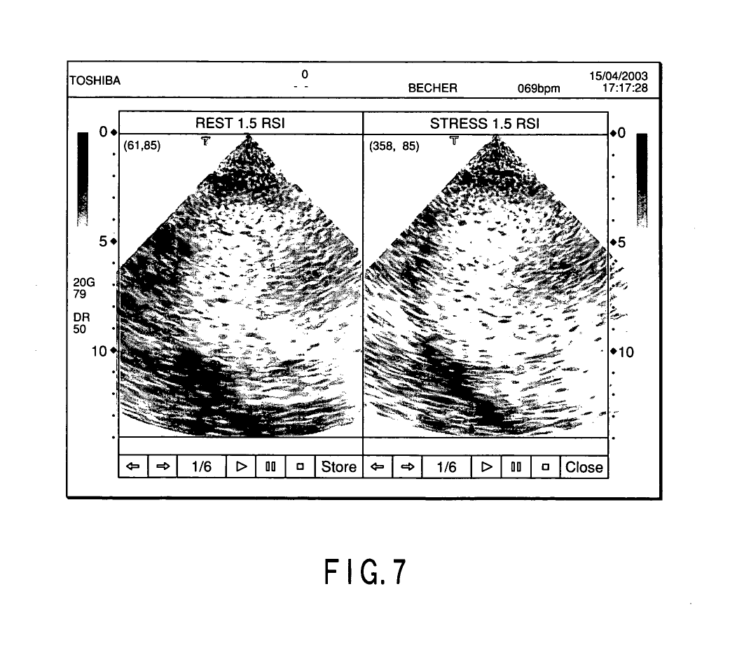

FIG. 7 is an illustration that indicates a still image (left) by the flash mode before stress synthetically displayed on the monitor 14 by the image comparison function and a still image (right) by the flash mode under stress. FIG. 7 corresponds to the display form shown in FIG. 2D. Physicians and others can efficiently and effectively carry out diagnosis of ischemic heart diseases by comparing perfusion brightness by myocardium by contrast agent in the same cross-section before stress and under stress.

In addition, under this image comparison screen state, it is possible to carry out measurement processing concerning each image (step S24). That is, pressing a switch for starting measurement function by the external input switch 13b calls a measurement function program from the software storage unit 28, and by being developed on memory not illustrated, the measurement function is started. The operator sets ROI (Region Of Interest) by the external input 13 such as track ball 13a, respectively, for each screen displayed. The control processor 25 uses the started measurement function to perform arithmetic on mean value, most frequent value, maximum value, standard deviation, distribution, etc. which are typically statistical values in relevant ROI using the started measurement function by the control processor 25, synthesizes by the image generation circuit 24 and displays on the monitor 14.

Then, whether or not image comparison should be executed for other image pickup phases is determined, and if executed, each processing of steps S21 to S24 is repeated. On the other hand, if it is not executed, image comparison processing is ended.

According to the foregoing configuration, the following effects can be obtained.

According to the ultrasonic diagnostic apparatus, in the event that images are acquired by the predetermined imaging mode in examination under stress, images acquired by the same imaging mode in examination before stress are read in accordance with control information and are displayed simultaneously with current images. Consequently, the operator can utilize images acquired in the corresponding imaging mode as appropriate reference images even when a complicated examination such as a stress examination for ischemic heart disease in which examinations before and after stress and multiple imaging modes are mixedly performed. As a result, in the examination by comparison evaluation of perfusion brightness in the same cross-section before stress and under stress, substantially identical cross sections can be objectively and conveniently chosen before and after stress.

In addition, according to the ultrasonic diagnostic apparatus, when multiple image pickup phases exist in the same imaging mode (that is, multiple picked up cross-sections exist), the image that corresponds to the same pickup phase is read and displayed as a reference image. Consequently, even if multiple image pickup phases exist, appropriate reference images can e chosen.

In addition, according to the ultrasonic diagnostic apparatus, by the image saving function, by the predetermined operation, control information concerning the image to be saved is automatically saved. Consequently, it is not necessary to carry out information control to properly select reference images and comparison images, and work burden of the operator can be reduced.

Furthermore, according to the ultrasonic diagnostic apparatus, only by choosing the imaging mode and image pickup phase, images concerning the same image pickup phase acquired by the same imaging mode between before stress and under stress can be automatically and simultaneously displayed. Consequently, images easy to be observed can be quickly and easily provided with less work burden.

### Second Embodiment

Next description will be made on the second embodiment of the present invention. The present embodiment utilizes minor programs (hereinafter, referred to as the “activities”), which are individual component elements of the examination procedure in the system that programs a series inspection procedures in advance and supports the physician's or engineer's examination procedure (workflow). This workflow system is described in detail by, for example, Jpn. Pat. Appln. KOKAI Publication No. 2001-137237.

In the present workflow system, by “image saving activity,” “image reference display activity,” and “image comparison display activity” as the activities, existing image related storage function, image reference function, and image comparison function are realized.

In this event, the “image saving activity” means a minor program for saving dynamic images or still images or both of varying imaging modes in the internal memory device 26 or external memory device 29. For example, in the event that processing is adopted not to separate multiple imaging modes but to store them as a series of information, it may be configured to carry out a processing to separate them in accord with the imaging modes ex post facto. In addition, by this image saving activity, control information including the path name of storage place of each image, file name or folder name, the imaging mode, the pickup phase, and other ancillary information, and information that correlates images acquired by the same imaging mode is created and saved in the internal memory device 26 or external memory device 29.

In addition, the “image reference/comparison display activity” is a minor program which chooses images (moving images or still images) of the same image pickup phase acquired by the mode same as the chosen image mode based on the control information and displays for reference on either left half or right. half of the monitor display. This image reference/comparison display activity has a preset to receive the output file to which the image saving activity outputs and to decide in advance which imaging mode is used and furthermore whether the reference display function or comparison display function should be used, and these can be set when a series of inspection procedure are edited. At the time of reference display function, it links to one designated image saving activity and at the time of comparison display function, it links to two image saving activities in the present case. The functions to link can be mounted to the inspection procedure editing program.

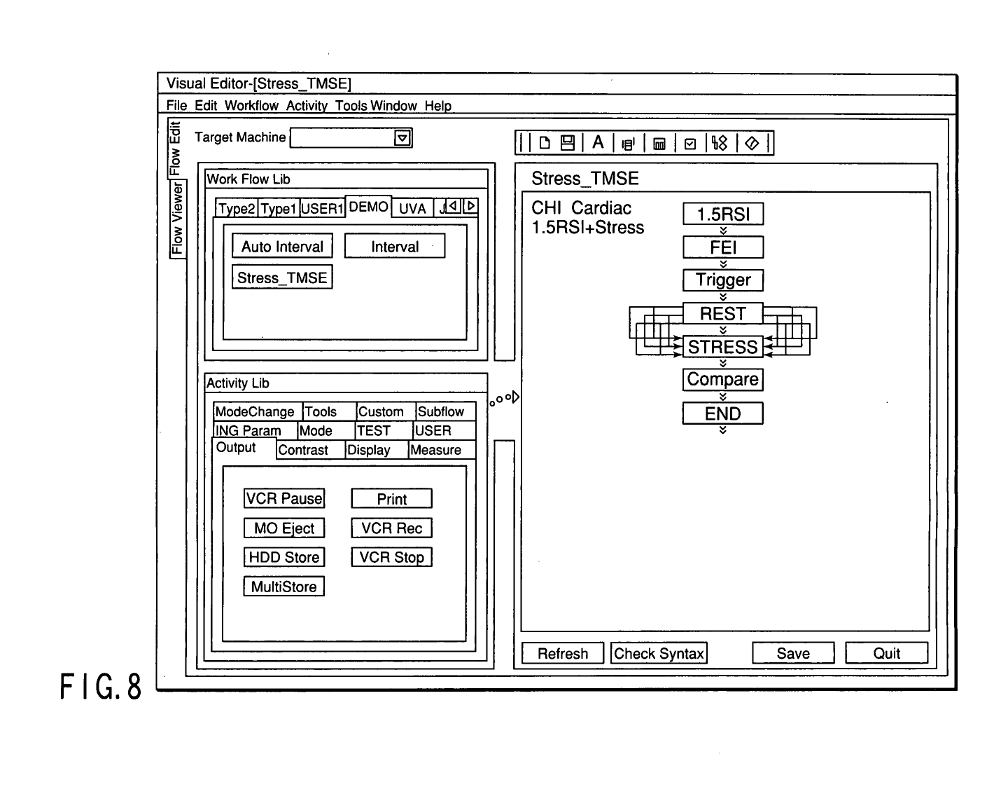

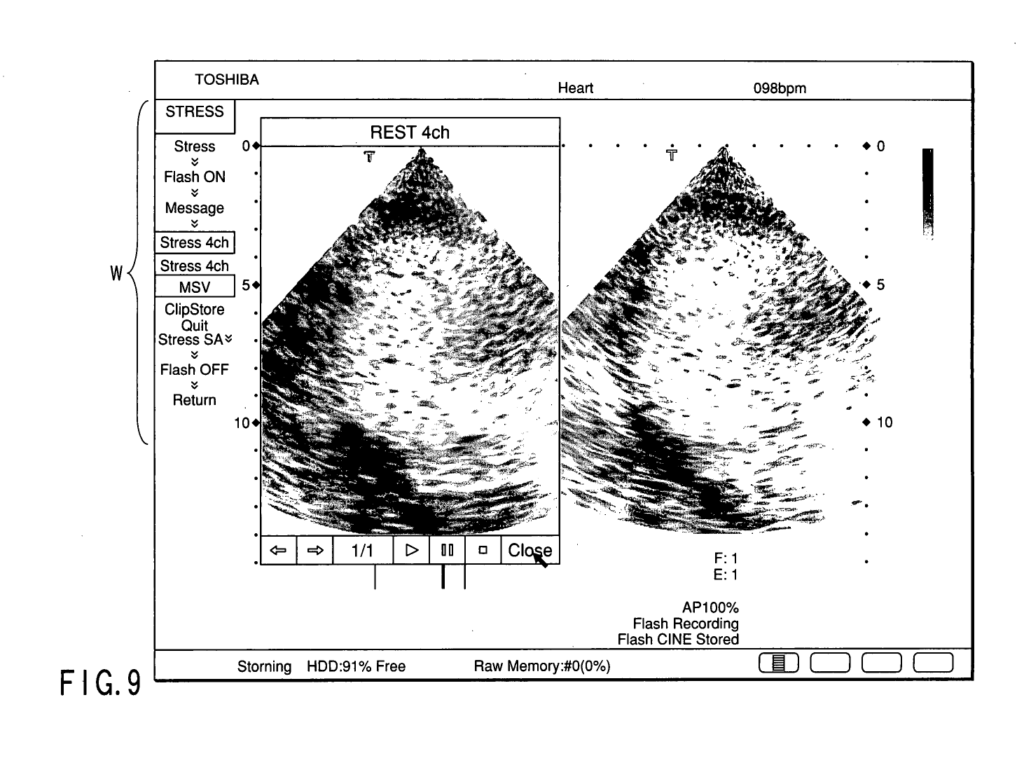

The workflow that contains each activity is created by the program, which edits the inspection procedure. FIG. 8 is an illustration that indicates one example of edit screen of workflow. Each activity is saved in the software storage unit 28. The operator inserts parts corresponding to each activity (for example, “REST”, “STRESS”, “Compare” in FIG. 9) in a predetermined place by drug-and-drop or double click, or other operations. By this kind of editing operation, a workflow including only the required number of activities needed is defined and by starting this workflow, an examination, which follows the optional procedure, can be executed.

FIG. 9 is an illustration that indicates one example of workflow W, which defines stress examination concerning ischemic heart diseases. As shown in the figure, the workflow W is displayed at a predetermined position (left side of the screen in the same figure) of the monitor 14 in the Window's format. In accordance with the examination flow defined in this workflow W, same as the content described in the first embodiment, scanning is carried out in two different modes, namely, the flash mode and monitor mode, and with images of each mode displayed in two screens, respectively, right and left of the monitor screen, contrast agent are continuously infused before stress and under stress. While multiple cross-sections, such as apical four-chamber view, apical two-chamber view, etc., are being observed in the monitor mode before stress and under stress, perfusion images of intramyocardial blood flow are acquired by high sound pressure intermittent transmission in the same cardiac time phase by an electrocardiograph synchronizer unit.

According to the foregoing second embodiment, by the operation using the operation support system, image pickup operations, etc. same as the first embodiment can be achieved.

In addition, because by the workflow, it is possible to define the processing covering the period before and after stress as a series of workflow system, burdens to the operator can be alleviated and efficiency of operation can be improved.

### Third Embodiment

Next, the third embodiment according to the present invention will be described. The present embodiment further improves the degree of freedom of diagnostic image selection in image comparison by carrying out ultrasound scan (volume scan) concerning the three-dimensional region in the contrast echo technique implemented in stress examination concerning ischemic heart diseases.

First of all, a configuration of an ultrasonic diagnostic apparatus 1 according to the present embodiment will be described. In FIG. 1, for the ultrasonic probe 12, a two-dimensional ultrasonic probe with piezoelectric vibrators arranged in a two-dimensional matrix is used. The transmitter-receiver unit 21 supplies the drive signal for volume scan to each of piezoelectric vibrators of the ultrasonic probe 12 in accordance with control signals from the control processor 25.

Each of the transmitter-receiver unit 21, B-mode processor unit 22, and Doppler processing unit 23 execute the above-mentioned processing to each echo signal obtained by volume scan.

The image generation circuit 24 creates volume data of B-mode images using the detection data received from the B-mode processing unit 22. In addition, the image generation circuit 24 creates volume data of blood flow information using the blood flow information received from the Doppler processing unit 23. Further, the image generation circuit 24 creates ultrasonic images, volume rendering images, etc. concerning optional cross-sections of various kinds of volume data.

The image memory 27 stores various kinds of volume data, ultrasonic images, etc. created in the image generation circuit 24.

Referring now to FIGS. 3 and 4, scan operation before stress and after stress of the ultrasonic diagnostic apparatus 1 will be described. The ultrasonic diagnostic apparatus 1 implements the volume scan in reperfusion image/flash image pickup processing of step S5. In addition, the ultrasonic diagnostic apparatus 1 implements the volume scan in reperfusion image/flash image pickup processing in step S18.

For example, in cross-section determination processing of step S3, step S15, etc., too, the volume scan may be implemented. In such a case, for the alignment image, tomographic images preset (or manually set) in the volume scan region or three-dimensional images obtained by volume scan can be adopted.

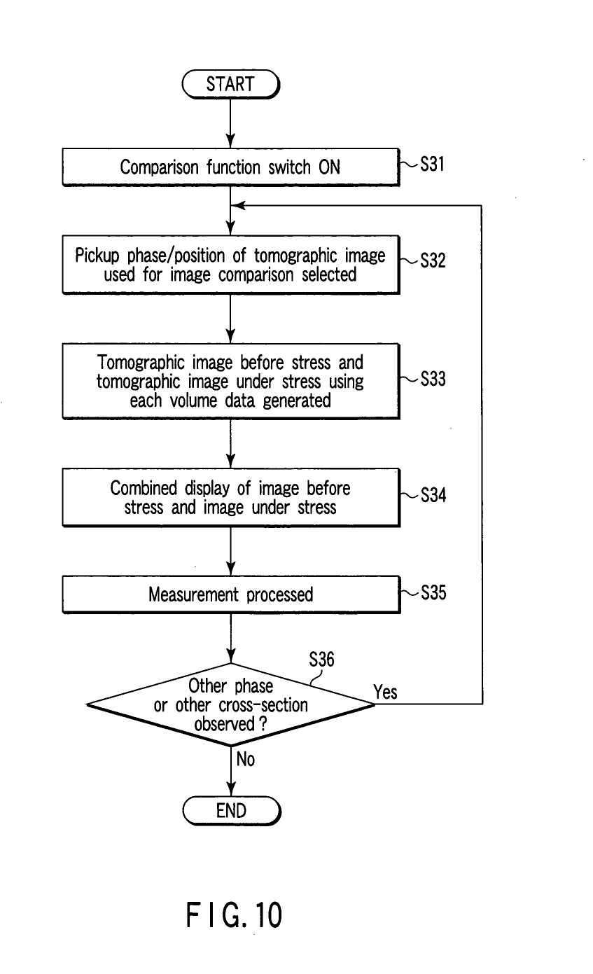

Now, description will be made on the image comparison function, which the ultrasonic diagnostic apparatus 1 possesses. FIG. 10 is a flow chart that indicates a flow of each processing executed in accordance with the image comparison function of the ultrasonic diagnostic apparatus 1. As shown in the same figure, first of all, pressing the image comparison function switch SW4 calls programs for comparison function from the software storage unit 28 in response to this operation, and under the control of the control processor 25, the image comparison function is started (step S31). Thereafter, desired image pickup phase subject to image comparison and tomographic images used for comparison before and after stress are set (position selection) (Step S32). In this event, short-axis cross-section of a predetermined level should be set.

When the image pickup phase subject to image comparison is chosen, volume data before stress and volume data under stress, which correspond to the image pickup phase chosen in accordance with the control information, are read. The image generation circuit 24 generates tomographic images before stress and under stress, respectively, in the selected position (step S33). Each of tomographic images generated is generated in the image generation circuit 24 so that the image by the flash mode before stress, for example, is displayed on the left half of the screen and the image by the flash mode under stress is displayed on the right half of the screen (Step S34) of the monitor 14. In addition, under the condition of this image comparison screen, measurement processing for each image is implemented as required (step S35).

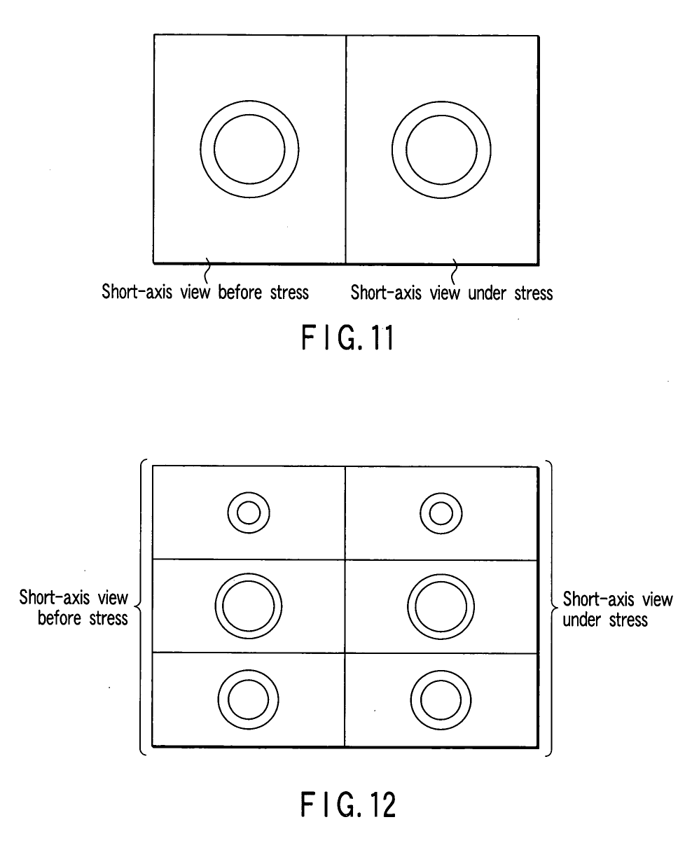

FIG. 11 is an illustration that indicates one example of tomographic images before and after stress displayed on the monitor 14 by the image comparison function. As shown in the figure, in the present image comparison function, without being bound by the comparison image (see FIG. 7) used for determining the cross-sectional position, the tomographic image (in the present case, short-axis view) chosen in step S32 is displayed.

In step S32, it is possible to choose multiple tomographic images with varying positions (for example, three short-axis views with varying levels). In such a case, short-axis views corresponding to each level using the volume before and after stresses are created, for example, in the form shown in FIG. 12.

According to the above-mentioned configuration, using the volume data obtained by volume scan, it is possible to choose optional tomographic image as a comparison image. Consequently, observers such as physicians can use the desired tomographic images for diagnosis in stress examinations related to ischemic heart diseases without being bounded to the position of images used to determine the cross-sectional position. In addition, by selecting multiple tomographic images with varying positions, the lesion can be observed from various cross sections. As a result, the degree of freedom of selecting diagnostic images in image comparison can be still more improved, contributing to improvement in the diagnostic quality.

The present invention shall not be limited to the above-mentioned embodiments as they are but in the working stages, the invention may be embodied by changing the component elements in various forms without departing from the spirit and scope thereof. Specific examples include the following:

(1) Each function according to the present embodiment can be achieved by installing programs to implement the relevant processing to computers such as workstations, etc. and deploying these on memory. In such a case, the program that allows the computer to implement the relevant technique can be stored in recording media such as magnetic disks (floppy (trademark) disks, hard disks, etc.), optical disks (CD-ROM, DVD, etc.), semiconductor memory, etc. and can be distributed.

(2) In each of the above-mentioned embodiments, cases of two kinds of examinations before stress and under stress, two kinds of imaging modes in the monitor mode and flash mode, and three kinds of image pickup phases that correspond to three kinds of cross sections of apical four-chamber view, apical two-chamber view, and parasternal short-axis view were described as examples. However, the number of imaging modes, etc. are merely illustrative and it is not intended to limit to these. For example, where the number of examinations exceeds three kinds, it may be configured to achieve each of the above functions between combinations of the predetermined two sets thereof, or images corresponding to each examination may be simultaneously displayed in such a manner as to be able to be simultaneously referred to or compared among examinations of three kinds or more.

(3) In each of the above-mentioned embodiments, it was configured to create control information and store separately from the image data to be saved. However, the invention shall not be limited to this but it may be configured to achieve the same functions by recording the control information as the ancillary information of images, or more specifically, by recoding the control information in a private tag of DICOM.

(4) In addition, a series of inspection procedures shown in the third embodiment can be achieved by the use of the workflow system shown in the second embodiment.

In addition, by properly combining multiple component elements disclosed in the above-mentioned embodiments, various inventions may be formed. For example, from all the component elements shown in embodiments, several component elements may be deleted. Furthermore, component elements related to different embodiments may be properly combined.

What is claimed is:

1. An ultrasonic diagnostic apparatus comprising:
an image acquisition unit which acquires a plurality of ultrasonic images by a first imaging mode which executes a first ultrasonic transmission that destroys a contrast agent and a second imaging mode which executes the second ultrasonic transmission that visualizes a process of flowing in of contrast agent without practically destroying the contrast agent in examination before stress and examination under stress of a subject implemented in time series;
a storage unit which stores a plurality of ultrasonic images in each examination and in each imaging mode;
an image generator unit which generates images acquired in the same imaging mode in the examination before stress as reference images in accordance with the stored ultrasonic images, when current images are acquired in the predetermined imaging mode in the examinations under stress; and
a display unit which simultaneously displays the reference image and the current images.

2. The ultrasonic diagnostic apparatus according to claim 1, wherein the image generator unit generates ultrasonic images of the same kind as the current images as the reference images when multiple kinds of ultrasonic images are acquired in each of imaging modes.

3. The ultrasonic diagnostic apparatus according to claim 1, further comprising:
a control information generator unit which generates control information for correlating ultrasonic images of the same kind by the same imaging mode, wherein
the storage unit stores the control information, and
the image generator unit generates the reference images in accordance with the control information.

4. The ultrasonic diagnostic apparatus according to claim 1, further comprising:
an image reproduction unit which simultaneously reproduces ultrasonic images of the same kind by the same imaging mode in accordance with the control information.

5. The ultrasonic diagnostic apparatus according to claim 1, wherein the image acquisition unit acquires the ultrasonic images as volume data by ultrasonic-scanning the three-dimensional region at least in the first imaging mode, and
the image generator unit generates the reference images which correspond to the predetermined cross-section in accordance with the volume data acquired in the first imaging mode in the examination before stress and generates the current images which correspond to the predetermined cross-section in accordance with the volume data acquired by the first imaging mode in the examination under stress.

6. An ultrasonic diagnostic apparatus comprising:
an image acquiring unit which acquires a plurality of ultrasonic images in accordance with a plurality of imaging modes in each of the examinations executed in time series;
a storage unit which stores the plurality of ultrasonic images in accord with examinations and imaging modes;
an image generator unit which generates images acquired in the same imaging mode in examinations executed in the past as reference images in accordance with the multiple ultrasonic images stored, when current images are acquired by the predetermined imaging modes in the examination under execution; and
a display unit which simultaneously displays the reference images and the current images.

7. The ultrasonic diagnostic apparatus according to claim 6, wherein the image generator unit generates ultrasonic images of the same kind as the current images as the reference images when multiple kinds of ultrasonic images are acquired in each of imaging modes.

8. The ultrasonic diagnostic apparatus according to claim 6, wherein at least one of the multiple imaging modes is either one of a two-dimensional imaging mode, a three-dimensional imaging mode, a color mode, a Doppler mode, a B mode, and an M mode.

9. An ultrasonic diagnostic apparatus control method, comprising:
acquiring a plurality of ultrasonic images in a first imaging mode which executes a first ultrasonic transmission to destroy a contrast agent and image the perfusion image condition and a second imaging mode which executes a second ultrasonic transmission to image the process in which the contrast agent flows in without practically destroying the contrast agent;
storing the plurality of ultrasonic images in accord with examinations and imaging modes;
generating images acquired by the same imaging mode in the examination before stress as reference images in accordance with the plurality of ultrasonic images stored when current images are acquired by the predetermined imaging mode in the examination under stress; and
displaying simultaneously the reference images and the current images.

10. The ultrasonic diagnostic apparatus control method according to claim 9, wherein ultrasonic images of the same kind as the current images are generated as the reference images when multiple kinds of ultrasonic images are acquired in each of imaging modes.

11. The ultrasonic diagnostic apparatus control method according to claim 9, further comprising generating control information for correlating ultrasonic images of the same kind by the same imaging mode, wherein
the control information is stored in the step of storing, and
the reference images are generated in accordance with the control information in the step of generating images.

12. The ultrasonic diagnostic apparatus control method according to claim 9, further comprising:
reproducing simultaneously ultrasonic images of the same kind by the same imaging mode in accordance with the control information.

13. The ultrasonic diagnostic apparatus control method according to claim 9, wherein
the ultrasonic images are acquired as volume data by ultrasonic-scanning the three-dimensional region at least in the first imaging mode, and
the reference images which correspond to the predetermined cross-section are generated in accordance with the volume data acquired in the first imaging mode in the examination before stress and the current images which correspond to the predetermined cross-section are generated in accordance with the volume data acquired by the first imaging mode in the examination under stress.

14. An ultrasonic diagnostic apparatus control method, comprising:
acquiring multiple ultrasonic images in accordance with multiple imaging modes in each of the multiple examinations executed in time series;
storing the a plurality of ultrasonic images in accord with examinations and imaging modes;
generating images acquired in the same imaging mode in examinations executed in the past as reference images in accordance with the stored ultrasonic images, when current images are acquired by the predetermined imaging modes in the examination under execution; and
displaying simultaneously the reference images and the current images.

15. The ultrasonic diagnostic apparatus control method according to claim 14, wherein ultrasonic images of the same kind as the current images are generated as the reference images when multiple kinds of ultrasonic images are acquired in each of imaging modes.

16. The ultrasonic diagnostic apparatus control method according to claim 14, further comprising generating control information for correlating ultrasonic images of the same kind by the same imaging mode, wherein
the control information is stored in the step of storing, and
the reference images are generated in accordance with the control information in the step of generating images.

17. The ultrasonic diagnostic apparatus control method according to claim 14, further comprising:
reproducing simultaneously ultrasonic images of the same kind by the same imaging mode in accordance with the control information.

18. The ultrasonic diagnostic apparatus control method according to claim 14, wherein at least one of the multiple imaging modes is either one of a two-dimensional imaging mode, a three-dimensional imaging mode, a color mode, a Doppler mode, a B mode, and an M mode.

19. The ultrasonic diagnostic apparatus control method according to claim 14, wherein the multiple imaging modes are determined by the subject's regions.

20. The ultrasonic diagnostic apparatus control method according to claim 14, wherein
the ultrasonic images are acquired as volume data by ultrasonically scanning in a flash mode in which a first ultrasonic transmission is executed to image the perfusion image condition by at least destroying the contrast agent, and
the reference image which corresponds to the predetermined cross-section is generated in accordance with the volume data acquired in the flash mode in the examination before stress, and at the same time, the current image which corresponds to the predetermined cross-section is generated in accordance with the volume data acquired in the flash mode in the examination under stress.

21. An ultrasonic diagnostic apparatus control method, comprising:
acquiring a first image according to a first imaging mode and a second image according to a second image respectively in a first examination;
storing the first image and the second image according to imaging mode;
displaying the first image and a third image simultaneously when the third image is acquired according to the first imaging mode in a second examination which is performed after the first examination;
displaying the third image and a fourth image simultaneously when the fourth image is acquired according to the second imaging mode in the second examination; and
displaying the second image and the fourth image simultaneously when the second examination is completed.
